SEQUENCE LISTING (24 sequences)

SEQ ID NO: 1 - DNA, 20 bp, Artificial Sequence
  bovine-specific mitochondrial cytochrome-b (mt-cyt-b) PCR forward primer
gtggactatggcaattgcta

SEQ ID NO: 2 - DNA, 19 bp, Artificial Sequence
  bovine-specific mitochondrial cytochrome-b (mt-cyt-b) PCR reverse primer
ctgaggcggattctcagta

SEQ ID NO: 3 - DNA, 23 bp, Artificial Sequence
  bovine-specific mitochondrial cytochrome-b (mt-cyt-b) fluorescein labeled FRET probe
acccgattcttcgctttccattt

SEQ ID NO: 4 - DNA, 14 bp, Artificial Sequence
  bovine-specific mitochondrial cytochrome-b (mt-cyt-b) LC-Red 640 labeled FRET probe
tccttccatttatc

SEQ ID NO: 5 - DNA, 17 bp, Artificial Sequence
  ovine-specific mitochondrial cytochrome-b (mt-cyt-b) PCR forward primer
gctaccctcacccgatt

SEQ ID NO: 6 - DNA, 21 bp, Artificial Sequence
  ovine-specific mitochondrial cytochrome-b (mt-cyt-b) PCR reverse primer
gagtaggtgaactatggcgag

SEQ ID NO: 7 - DNA, 14 bp, Artificial Sequence
  ovine-specific mitochondrial cytochrome-b (mt-cyt-b) fluorescent labeled FRET probe
gcctttcactttat

SEQ ID NO: 8 - DNA, 16 bp, Artificial Sequence
  ovine-specific mitochondrial cytochrome-b (mt-cyt-b) LC-Red 640 labeled FRET probe
tcccattcatcatcgc

SEQ ID NO: 9 - DNA, 35 bp, Artificial Sequence
  chloroplast RNA polymerase beta-subunit (rpobeta) fluorescein labeled FRET probe
ccataagggaatttctatatatgccaggactataa

SEQ ID NO: 10 - DNA, 29 bp, Artificial Sequence
  chloroplast RNA polymerase beta-subunit (rpobeta) LC-Red 705 labeled FRET probe
tcagattgggggagaaggtcggaattagc

SEQ ID NO: 11 - DNA, 17 bp, Artificial Sequence
  goat-specific mitochondrial cytochrome-b (mt-cyt-b) PCR forward primer
acccgattcttcgcctt

SEQ ID NO: 12 - DNA, 18 bp, Artificial Sequence
  goat-specific mitochondrial cytochrome-b (mt-cyt-b) PCR reverse primer
tggggttgttcgatcctg

SEQ ID NO: 13 - DNA, 19 bp, Artificial Sequence
  goat-specific mitochondrial cytochrome-b (mt-cyt-b) fluorescein labeled FRET probe
agccctcgccatagtccac

SEQ ID NO: 14 - DNA, 13 bp, Artificial Sequence
  goat-specific mitochondrial cytochrome-b (mt-cyt-b) LC-Red 705 labeled FRET probe
gctcttcctccac

SEQ ID NO: 15 - DNA, 17 bp, Artificial Sequence
  horse-specific mitochondrial cytochrome-b (mt-cyt-b) PCR forward primer
gccacccttacccgatt

SEQ ID NO: 16 - DNA, 21 bp, Artificial Sequence
  horse-specific mitochondrial cytochrome-b (mt-cyt-b) PCR reverse primer
taaatgtacgactaccagggc

SEQ ID NO: 17 - DNA, 13 bp, Artificial Sequence
  horse-specific mitochondrial cytochrome-b (mt-cyt-b) fluorescein labeled FRET probe
gctttccacttca

SEQ ID NO: 18 - DNA, 14 bp, Artificial Sequence
  horse-specific mitochondrial cytochrome-b (mt-cyt-b) LC-Red 705 labeled FRET probe
ctacccttcatcat

SEQ ID NO: 19 - DNA, 19 bp, Artificial Sequence
  pig-specific mitochondrial cytochrome-b (mt-cyt-b) PCR forward primer
tcacacgattcttcgcctt

SEQ ID NO: 20 - DNA, 22 bp, Artificial Sequence
  pig-specific mitochondrial cytochrome-b (mt-cyt-b) PCR reverse primer
gtgcaggaataggagatgtacg

SEQ ID NO: 21 - DNA, 14 bp, Artificial Sequence
  pig-specific mitochondrial cytochrome-b (mt-cyt-b) fluorescein labeled FRET probe
tttatcctgccatt

SEQ ID NO: 22 - DNA, 15 bp, Artificial Sequence
  pig-specific mitochondrial cytochrome-b (mt-cyt-b) LC-Red 705 labeled FRET probe
tcattaccgccctcg

SEQ ID NO: 23 - DNA, 25 bp, Artificial Sequence
  chloroplast RNA polymerase beta-subunit (rpobeta) PCR forward primer
gtgatcaatcaaatattgctaagtc

SEQ ID NO: 24 - DNA, 21 bp, Artificial Sequence
  chloroplast RNA polymerase beta-subunit (rpobeta) PCR reverse primer
acccatagctgatgatagaac

(19) United States
(12) Patent Application Publication
(10) Pub. No.: US 2007/0269822 A1
(43) Pub. Date: Nov. 22, 2007

(54) Detection of contaminating DNA via apmplification of sequences of less than 100 bp

(75) Inventors: James Cullor, Woodland, CA (US); Wayne Smith, Fairfield, CA (US); Bennie Osburn, Wilton, CA (US); Gabriel Rensen, Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, Oakland, CA (US)

(21) Appl. No.: 11/701,596

(22) Filed: Feb. 2, 2007

Related U.S. Application Data
(60) Provisional application No. 60/764,880, filed on Feb. 3, 2006.

(51) Int. Cl. C12Q 1/68; C07H 21/00

(52) U.S. Cl. 435/6; 536/22.1; 536/25.3

(57) ABSTRACT

The present invention provides methods, compositions and kits for amplifying, measuring, and/or detecting contaminating DNA (e.g., ruminant and non-ruminant) in samples by amplification and/or detection of a target DNA of less than 100 base pairs in the samples.

### CROSS-REFERENCES TO RELATED APPLICATIONS

This application claims the benefit of U.S. Provisional Patent Application No. 60/764,880, filed Feb. 3, 2006, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Bovine spongiform encephalopathy (BSE) or “Mad Cow” disease was first recognized in Great Britain in 1986 and spread to countries on the European continent (see, e.g., Anderson et al., Nature 382:779-88 (1996)). Subsequent epidemiological studies have identified rendered material from scrapie infected sheep into bovine feeds as the most probable initial cause of BSE. The pathogenic agent of BSE, i.e., prions were spread to cows from the rendered offal. BSE was further propagated by the inclusion of rendered bovine meat and bone meal (BMBM) as a component of animal feeds (see, e.g., Wilesmith et al., Vet Rec. 123:112-3 (1988)). BSE has now been identified in the United Kingdom, Europe, Japan, and North America, including Canada and the United States (see, e.g., Normile, Science, 303:156-157 (2004)).

In 1997, in response to epidemiologic evidence regarding the transmission of BSE, the Food and Drug Administration of the United States (FDA) prohibited the incorporation of certain mammalian tissues (e.g., tissue derived from the CNS, and intestinal tissue) in ruminant feed (see, e.g., 62(108) Federal Register 30935-78 (Jun. 5, 1997)). Products believed to pose a minimal risk, including blood, blood products, gelatin, milk and milk products, protein deprived solely from swine or equine sources and inspected meat products offered for human consumption were initially exempted from the ban. In January of 2004, the USDA prohibited the incorporation of “specified risk materials,” i.e., skull, brain, trigeminal ganglia, eyes, vertebral column, spinal cord, and dorsal root ganglia of cattle 30 months and older, as well as tonsils and distal ileum of the small intestine from cattle of any age into any human food, including any food that is likely to enter the human food supply. In the same month, the FDA extended the ban to mammalian blood and blood products, uneaten meat and other scraps from restaurants from ruminant feed. The European Union has banned use of any mammalian meat and bone meal from any ruminant feed.

In addition, the FDA has advised that bovine derived materials from animals born in or residing in countries where BSE had occurred should not be used to manufacture FDA-regulated products intended for administration to humans (including, e.g., vaccines). The FDA has also recommended that the use of high-risk cattle-derived protein be avoided in the manufacture of cosmetics

Currently, estimates of compliance are based on an honor system accompanied by signatures and FDA site visits in which manufacturing protocols and record keeping are checked. The tests for verification currently available for determining the presence of ruminant source proteins in animal feed is a time consuming microscopic examination method (Tartaglia et al., J Food Prot. 61(5):513-518 (1998)) which has a lower limit of detection greater than 5% by weight of feed or immunological assays with a reported detection limit of 1%-5% by weight (“Reveal®” Neogen Corp., Lansing Mich.).

Since the initial bans were implemented, development of methods for extracting and identifying banned additives in samples (e.g., ruminant feed, pet food, cosmetics, human food, and nutraceuticals) has been given a great deal of attention by researchers. For example, Tartaglia et al., J Food Prot. 5:513-518 (1998); Wang et al., Mol. Cell Probes 1:1-5 (2000); and Kremar and Rencova, J Food Prot. 1:117-119 (2001) describe methods of extraction and identification of bovine mitochondrial DNA. Myers et al., J Food Prot. 4:564-566 (2001) compared methods of nucleic acid extraction. However, none of these methods address the issue of false negatives (i.e. lack of detection of DNA material of bovine sources) due to the break down of DNA into smaller fragments upon sample processing during product manufacture.

The application of the polymerase chain reaction (PCR) of mitochondrial DNA (mtDNA) has been investigated for detecting the presence of bovine contamination in ruminant feed (Tartaglia et al., J Food Prot. 61(5):513-518 (1998)). However, the procedure failed to detect contamination levels below 0.125% by weight, and required an overnight incubation step. The investigators also suggested an additional step utilizing restriction endonuclease analysis of the amplified product to insure the specificity of the amplified product.

PCR is a highly specific and sensitive analytical method; however the reliability and sensitivity of PCR can be heavily dependent on a number of factors. Factors such as the DNA extraction methodology can influence both the DNA yield and the levels of PCR inhibitors present in the final sample. The size and abundance of the DNA target can also affect the reliability and the limit of detection of the PCR assay. (Gizzie et al. Rev Sci Tech. (1):311-31 (2003).

The limit of detection of PCR based assays can be highly influenced by the sample size, the method of DNA extraction and the rendering conditions that the MBM has been exposed to. Rendering conditions (e.g., temperature, pressure, and pH) have been reported to be a major limiting factor for the limit of detection of PCR based methods, e.g., by degrading and fragmenting the DNA target. Sterilization temperatures used during the rendering process can range between 118° C.-143° C., which can degrade and fragment the DNA targets (Kirstein et al., Modern Rendering: Benefits for the Feed Industry. II Latin American Rendering Products Nutrition Conference. Guadalajara, Jalasco, Mexico. June 19-20, 2002). European rendering temperatures are usually on the higher side and rendering temperatures used in the United States are usually on the lower end. In addition to sterilization temperatures, rendering processes may also use acids or bases which have a great damaging effect on DNA as well (von Holst et al., Overview of methods for the detection of species specific proteins in feed intended for farmed animals: September 2004. European Commission Directorate General Joint Research Centre).

Previous species-specific PCR methods have been developed for multiple species detection and differentiation in animal feed (Myers et al. J Food Prot. 66:1085-1089 (2003)). However reported methods which describe species specific PCR detection of ovine have not been evaluated on commercially rendered LMBM spiked into compound cattle feed. The methods were either evaluated on their ability to amplify ovine DNA isolated from blood (Myers et al. J Food Prot. 66:1085-1089 (2003)) or pure LMBM that was rendered in the laboratory and not commercially rendered LMBM and not spiked into compound cattle feed (Lahiff et al. Mol. Cel. Probes 15:27-35 (2002)).

In the United States, the FDA has developed a PCR based assay for detecting BMBM in cattle feed which underwent a validation study with a reported sensitivity of 0.125% wt/wt BMBM in cattle feed with an overall rate of false negative results of 0.83% (Myers et al. J Food Prot. 64:564-566 (2001)). However the amplicon size is 270 bp, which may either make the assay ineffective or substantially decrease the sensitivity when applied to European rendered BMBM.

False negative results which fail to detect the presence of banned protein (i.e., ruminant and non-ruminant) in the animal food supply, the human food supply, vaccines, nutraceuticals, or cosmetics, could lead to the contamination of these substances with the banned ruminant protein, either directly or indirectly. Such contamination could have a significant adverse impact on public health by increasing the risk of BSE. In addition, the higher risk of contamination has potentially devastating effects on the food, cosmetic, and vaccine industries by drastically increasing the costs associated with monitoring their products ruminant material. More sensitive tests to detect contaminating material (i.e., ruminant and non-ruminant)in any food, vaccines, or cosmetics before they enter the food, vaccine, or cosmetic would both increase the efficiency of monitoring food, vaccines, or cosmetics for contaminating material (i.e., ruminant and non-ruminant) and greatly reduce the risk of BSE to the general public.

Thus, there is a need in the art for additional methods and compositions for detecting contaminating DNA (i.e., ruminant and non-ruminant). In particular, there is a need for more sensitive and accurate methods for detecting contaminating DNA (e.g., ruminant and non-ruminant), which reduces and/or eliminates false negatives. The present invention addresses these and other needs.

### BRIEF SUMMARY OF THE INVENTION

The present invention provides methods and kits for amplifying, measuring and/or detecting contaminating DNA (e.g., ruminant or non-ruminant) by amplifying and/or detecting a target nucleic acid sequences of about 100 base pairs or less in the samples.

One embodiment of the invention provides a method of amplifying contaminating DNA in a sample (e.g., of an animal feed, an animal feed component, a cosmetic, a nutraceutical, a vaccine, a colloidal infusion fluid, or combinations thereof) by amplifying nucleic acid from the sample using a pair of primers designed to amplify a target DNA sequence of less than about 100 base pairs, thereby amplifying the contaminating DNA (e.g., ruminant and non-ruminant) present in the sample. In some embodiments, the methods further comprise detecting the amplified contaminating DNA (e.g., ruminant and non-ruminant). In some embodiments, the contaminating DNA (e.g., ruminant and non-ruminant) being detected is from a cow, a sheep, a goat, an elk, a deer, horse, swine, and combinations thereof. In some embodiments, the contaminating DNA (e.g., ruminant and non-ruminant) comprises a mitochondrial DNA sequence (e.g., cytochrome c, cytochrome b, 12S RNA, ATPase subunit 8, ATPase subunit 6, ATP synthetase, subunit 8, and subsequences thereof). In some embodiments, the primer pairs comprise the sequences set forth in SEQ ID NOS: 1 and 2; SEQ ID NOS: 5 and 6; SEQ ID NOS: 11 and 12; SEQ ID NOS: 15 and 16; or SEQ ID NOS: 19 and 20. In some embodiments, the sample is an animal feed (e.g., bovine tallow, milk or a fraction thereof). In some embodiments, the animal feed is cattle feed (e.g., comprising about 0.5% to about 30%, about 0.75 % to about 20%, or about 1% bovine tallow). In some embodiments, the methods further comprise detecting the amplified product (e.g., by direct detection of the amplified product or by contacting an oligonucleotide probe with the amplified product and detecting the complex formed by the probe and the amplified product). In some embodiments the oligonucleotide probe comprises a detectable label (e.g. fluorescent dye such as flourescein). In some embodiments, a pair of labeled oligonucleotide probes (e.g., SEQ ID NOS: 3 and 4; SEQ ID NOS: 7 and 8; SEQ ID NOS: 13 and 14, SEQ ID NOS: 17 and 18; or SEQ ID NOS: 21 and 22) is contacted with the amplified product. In some embodiments, the oligonucleotide probe comprises the sequence set forth in SEQ ID NOS: 3, 4, 7, 8, 13, 14, 17, 18, 21, or 22.

Another embodiment of the invention also provides a kit for detecting contaminating DNA (e.g., ruminant and non-ruminant). The kits typically comprise at least one pair of primers, and instructions for use. In some embodiments, the kits further comprising a second pair of primers. In some embodiments, the kits further comprise a probe or pair of probes for detecting the amplified contaminating DNA (e.g., ruminant and non-ruminant).

A further embodiment of the invention comprises isolated nucleic acids comprising the nucleic acid sequences set forth in SEQ ID NOS:1, 2, 3, 4, 5, 6, 7, 8; 11, 12, 13, 14, 15, 16, 17, 18, 19, 20, 21, or 22.

The compositions and methods of the present invention are described in greater detail below.

### BRIEF DESCRIPTION OF THE DRAWINGS

FIG. 1 depicts data from melting point analysis of the amplified products described in Example 2.

FIG. 2 depicts amplification curves detected in the F3 channel of the Roche LightCycler 1.5 generated from the internal control plant rpoβ primers and probes tested on DNA extracted from cattle feed and feed ingredients.

### BRIEF DESCRIPTION OF THE SEQUENCES

SEQ ID NO:1 sets forth a bovine-specific primer sequence.

SEQ ID NO:2 sets forth a bovine-specific primer sequence.

SEQ ID NO:3 sets forth a bovine-specific probe sequence.

SEQ ID NO:4 sets forth a bovine-specific probe sequence.

SEQ ID NO:5 sets forth an ovine-specific primer sequence.

SEQ ID NO:6 sets forth an ovine-specific primer sequence.

SEQ ID NO:7 sets forth an ovine-specific probe sequence.

SEQ ID NO:8 sets forth an ovine-specific probe sequence.

SEQ ID NO:9 sets forth an rpoβ-specific probe sequence.

SEQ ID NO:10 sets forth an rpoβ-specific probe sequence.

SEQ ID NO: 11 sets forth a goat-specific primer sequence.

SEQ ID NO:12 sets forth a goat-specific primer sequence.

SEQ ID NO:13 sets forth a goat-specific probe sequence.

SEQ ID NO:14 sets forth a goat-specific probe sequence.

SEQ ID NO: 15 sets forth a horse-specific primer sequence.

SEQ ID NO: 16 sets forth a horse-specific primer sequence.

SEQ ID NO: 17 sets forth a horse-specific probe sequence.

SEQ ID NO: 18 sets forth a horse-specific probe sequence.

SEQ ID NO: 19 sets forth a pig-specific primer sequence.

SEQ ID NO: 20 sets forth a pig-specific primer sequence.

SEQ ID NO: 21 sets forth a pig-specific probe sequence.

SEQ ID NO: 22 sets forth a pig-specific probe sequence.

SEQ ID NO: 23 sets forth a rpoβ-specific primer sequence.

SEQ ID NO: 24 sets forth a rpoβ-specific primer sequence.

### DETAILED DESCRIPTION OF THE INVENTION

### I. INTRODUCTION

The present invention provides methods and kits for amplifying, measuring and/or detecting contaminating DNA (e.g., ruminant and non-ruminant) in a sample (e.g., of an animal feed, an animal feed component, a cosmetic, a nutraceutical, a vaccine, a colloidal infusion fluid, or combinations thereof). In some embodiments, the invention provides methods for amplifying, measuring and/or detecting contaminating DNA (e.g., ruminant and non-ruminant) in animal feed or animal feed components. The present invention is based on the discovery that methods utilized to process a sample (e.g., a sample such as an animal feed, a cosmetic, a nutraceutical, or a vaccine that is being tested for the presence of contaminating DNA (e.g., ruminant and non-ruminant)) interfere with amplification reactions for detecting contaminating DNA (e.g., ruminant and non-ruminant) in the sample by way of causing degradation of DNA in the sample. The inventors have discovered that using primers designed to amplify DNA sequences of 100 base pairs or fewer improve the consistency and sensitivity of amplification reactions for detecting contaminating DNA (e.g., ruminant and non-ruminant).

### II. DEFINITIONS

A “sample” as used herein refers to a sample of any source which is suspected of containing contaminating polypeptides or nucleic acids encoding a contaminating polypeptide. Contaminating polypeptides or nucleic acids are understood to be polypeptides or nucleic acids that are not normally contained in the sample. These samples can be tested by the methods described herein and include, e.g., animal feed and feed components, ruminant feed and feed components, pet food, cosmetics, human food, nutraceuticals, vaccines, or colloidal infusion fluids. A sample can be from a laboratory source or from a non-laboratory source. A sample may be suspended or dissolved in liquid materials such as buffers, extractants, solvents and the like. Samples also include animal and human body fluids such as whole blood, blood fractions, serum, plasma, cerebrospinal fluid, lymph fluids, milk; and biological fluids such as cell extracts, cell culture supernatants; fixed tissue specimens; and fixed cell specimens.

“Mammal as used herein refers to any warm-blooded animal (of the class Mammalia), such as a cow, a sheep, a horse, a goat, a deer, an elk, a pig or swine, a dog, a cat, a rat, a whale, or a human, the female of which produces milk to feed the young.

“Ruminant” as used herein refers to a mammal of the suborder Ruminantia and having a stomach divided into multiple compartments (i.e., a rumen, a reticulum, an omasum, and an abomasum) and capable of digesting cellulose. Examples of ruminants include, e.g., cows, sheep, horses, goats, deer, elk, buffalo, bison, llamas, alpacas, dromedaries, camels, yaks, reindeer, giraffes and the like.

“Animal feed” and “animal feed component” as used herein refers to any composition or portion thereof that supplies nutrition to an animal. General components of animal feed include, for example, protein, carbohydrate, and fat. Specific components of animal feed include, for example, corn, beef tallow, blood and/or fractions thereof, milk and/or fractions thereof, molasses/sugar (e.g., raw or processed sugar, molasses from beets, sugar cane and citrus, and combinations thereof), carrots, candy bars, grains (e.g., wheat, oats, barley, triticale, rice, maize/corn, sorghum, rye, and combinations thereof), processed grain fractions (e.g., pollard, bran, millrun, wheat germ, brewers grain, malt combings, biscuits, bread, hominy, semolina, and combinations thereof), pulses/legumes (e.g., succulent or mature dried seed and immature pods of leguminous plants, including for example, peas, beans, lentils, soya beans, and lupins, and combinations thereof), oil seeds (e.g., cotton seed, sunflower seed, safflower seed, rape/canola seed, linseed, and sesame seed, and combinations thereof); plant protein meals (e.g., oilseed meals, peanut meal, soya bean meal, copra meal, palm kernel meal, and combinations thereof); fruit by-products (e.g., citrus pulp, pineapple pulp, pome fruit pomace, grape marc, grape pomace, and combinations thereof), pasture (e.g., grass and legume pastures and mixed grass/legume pastures), fodder (e.g., seeds, hay, silage and straw of legumes, grasses and cereals, sugar cane tops, and combinations thereof), forage (e.g., cereal forage, oilseed forage, legume forage, and combinations thereof), alfalfa (e.g., fresh, dried, mid bloom, and combinations thereof), barley grain, dried beet pulp, bluegrass, brewer's grains (e.g., wet, dried, and combinations thereof), Brome grass, Late Brome grass hay, Citrus pulp (e.g., dried, silage, and combinations thereof), clover (e.g., hay, silage, and combinations thereof), coconut meal, corn (e.g., cobs, ears, grain, silage, and combinations thereof), corn gluten feed, cottonseed (e.g., hulls, whole, meal, and combinations thereof), dried distiller's grain, fish meal, hominy feed, lamb meal, Lespedeza (e.g., fresh, hay, and combinations thereof), linseed meal, meat and bone meal (e.g., from cattle, sheep, goats, poultry, and combinations thereof), milk (fresh, dried, skimmed, and combinations thereof), millet, napier grass, orchard grass, peanut meal; natural sausage casings, foods containing “binders” comprising bovine collagen. Animal feed can also include supplemental components, such as, for example, minerals, vitamins, and nutraceuticals. Animal feed includes, for example, cattle feed, sheep feed, goat feed, dog feed, cat feed, deer feed, elk feed, and the like.

“Animals” or “animal” as used herein refers to any vertebrate organism. Animals include mammals, avians, amphibians, reptiles, ruminants, primates (e.g., humans, gorillas, and chimpanzees). Animals include domesticated animals (e.g., cattle, sheep, goats, pigs, chickens, ducks, turkeys, geese, quail, guinea hens, cats, and dogs) as well as undomesticated animals (e.g., elk, deer, reindeer, and giraffes). Animals may in the wild (i.e., in their native environments) or may be maintained in zoological parks. Other animals within the definition used herein include, for example, elephants, rhinoceroses, hippopotami, lions, tigers, bears, cougars, pumas, bobcats, and the like.

A “cosmetic” or “cosmeceutical” as used herein refers to any compound intended to be rubbed, poured, sprinkled, or sprayed on, introduced into, or otherwise applied to the human body for cleansing, beautifying, promoting attractiveness, or altering the appearance. Exemplary types of cosmetics include, e.g., skin conditioning agents, emollients, binders, and hair and nail conditioning agents. Exemplary cosmetics include, e.g., skin moisturizers (including, e.g., body lotions, skin lotions, and anti-wrinkle creams), skin cleansers, acne care products (including, e.g., skin moisturizers, skin cleansers, skin toners, and concealers) perfumes, lip moisturizers, lip balms, lipsticks, fingernail polishes, eye and facial makeup preparations, shampoos, hair conditioners, permanent waves, hair dyes, toothpastes, collagen implants, and deodorants, as well as any material intended for use as a component of a cosmetic product.

A “nutraceutical” as used herein refers to any substance that is a food or a part of a food and provides medical or health benefits, including the prevention and treatment of disease. Nutraceuticals include, e.g., isolated nutrients, dietary supplements and specific diets to genetically engineered designer foods, herbal products, and processed foods such as cereals, soups and beverages, a product isolated or purified from foods, and generally sold in medicinal forms not usually associated with food and demonstrated to have a physiological benefit or provide protection against chronic disease. Nutraceuticals also include any food that is nutritionally enhanced with nutrients, vitamins, or herbal supplements. Exemplary nutraceuticals include nutritional supplements such as, e.g., amino acids (including, e.g., Tyrosine, Tryptophan); oils and fatty acids (including, e.g., Linoleic acid and Omega 3 oils); minerals/coenzymes/trace elements (including, e.g., Iron, Coenzyme Q10, Zinc); vitamins (including, e.g., Ascorbic acid, Vitamin E); Protein (whey) powders/drinks; plant based/herbs (including, e.g., alfalfa, phytonutrients, saw palmetto); Herbal and Homeopathic remedies (including, e.g., Leopard's bane, St John's wort; Colitis treatments (including, e.g., those that contain bovine colostrums such as enemas); arthritis treatments (including, e.g., those that contain bovine glucosamine-chondroitin); joint cartilage replacements (including, e.g., those that contain bovine cartilage); digestive aids (bile salts, garlic oils); and weight management products (including, e.g., those that contain bovine proteins such as collagen, gelatin and whey protein).

A “vaccine” as used herein refers to a preparation comprising an infectious or immunogenic agent which is administered to stimulate a response (e.g., and immune response) that will protect the individual to whom it is administered from illness due to an infectious agent. Individuals to whom vaccines may be administered include any animals as defined herein. Vaccines include therapeutic vaccines given after infection and intended to reduce or arrest disease progression as well as preventive (i.e., prophylactic) vaccines intended to prevent initial infection. Infectious agents used in vaccines may be whole-killed (inactive), live-attenuated (weakened) or artificially (e.g. recombinantly) manufactured bacteria, viruses, or fungi. Exemplary vaccines include, e.g., E. coli Bacterin J5 strain (Upjohn), UltraBac 7 (Clostridum Chauvoei-Septicum-Novyi-Sordellii-Perfringens Types C&D Bacterin-Toxoid) (Pfizer), Spirovav (Leptospira Hardjo Bacterin) (Pfizer), Leptoferm-5 (Leptospira Canicola-Grippotyphosa-Hardjo-Icterohaemorrhagiae-Pomona Bacterin) (Pfizer), ScourGuard 3 (Bovine Rota-Coronavirus—Killed Virus) Clostridium Perfringens Type C-E. coli Bacterin-Toxoid) (Pfizer), Bovi-Shield Gold (Bovine Rhinotracheitis-Virus Diarrhea-Parainfluenza-Respiratory Syncytial Virus Vaccine Modified Live Virus) Leptospira Canicol-Grippotyphosa-Hardjo-Icterohaemorrhagiae-Pomona Bacterin (Pfizer), Defensor 3 Rabies Vaccine killed virus (Pfizer), and Vanguard Plus 5 Canine Distemper-Adenovirus Type 2-Coronavirus-Parainfluenza-Parvovirus Vaccine Modified Live killed Virus Leptospira Bacterin (Pfizer).

A “colloidal infusion fluid” as used herein refers to a fluid that when administered to a patient, can cause significant increases in blood volume, cardiac output, stroke volume, blood pressure, urinary output and oxygen delivery. Exemplary colloidal infusion fluids include, e.g., plasma expanders. Plasma expanders are blood substitute products useful for maintaining patients' circulatory blood volume during surgical procedures or trauma care hemorrhage, acute trauma or surgery, burns, sepsis, peritonitis, pancreatitis or crush injury. Exemplary plasma expanders include, e.g., albumin, gelatin-based products such as Gelofusine®, and collagen-based products. Plasma expanders may be derived from natural products or may be recombinantly produced.

“PCR inhibitor” as used herein refers to any compound that affects a PCR amplification process, i.e., by interfering with any portion the amplification process itself or by interfering with detection of the amplified product. The PCR inhibitor may physically, i.e., mechanically interfere with the PCR reaction or detection of the amplified product. Alternatively, the PCR inhibitor may chemically interfere with the PCR reaction or detection of the amplified product.

An “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

“Amplifying” refers to submitting a solution to conditions sufficient to allow for amplification of a polynucleotide if all of the components of the reaction are intact. Components of an amplification reaction include, e.g., primers, a polynucleotide template, polymerase, nucleotides, and the like. Thus, an amplifying step can occur without producing a product if, for example, primers are degraded.

“Detecting” as used herein refers to detection of an amplified product, i.e., a product generated using the methods known in the art. Suitable detection methods are described in detail herein. Detection of the amplified product may be direct or indirect and may be accomplished by any method known in the art. The amplified product can also be measured (i.e., quantitated) using the methods known in the art.

“Amplification reagents” refer to reagents used in an amplification reaction. These reagents can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

The term “primer” refers to a nucleic acid sequence that primes the synthesis of a polynucleotide in an amplification reaction. Typically a primer comprises fewer than about 100 nucleotides and preferably comprises fewer than about 30 nucleotides. Exemplary primers range from about 5 to about 25 nucleotides. The “integrity” of a primer refers to the ability of the primer to primer an amplification reaction. For example, the integrity of a primer is typically no longer intact after degradation of the primer sequences such as by endonuclease cleavage.

A “probe” or “oligonucleotide probe” refers to a polynucleotide sequence capable of hybridization to a polynucleotide sequence of interest and allows for the detecting of the polynucleotide sequence of choice. For example, “probes” can comprise polynucleotides linked to one or more fluorescent or radioactive reagents, thereby allowing for the detection of these reagents.

The term “subsequence” refers to a sequence of nucleotides that are contiguous within a second sequence but does not include all of the nucleotides of the second sequence.

A “target” or “target sequence” refers to a single or double stranded polynucleotide sequence sought to be amplified in an amplification reaction. Two target sequences are different if they comprise non-identical polynucleotide sequences. The target sequences may be mitochondrial DNA or non-mitochondrial DNA. Suitable mitochondrial target sequences include, for example, cytochrome B, cytochrome C, 12S RNA, ATPase subunit 8, ATPase subunit 6, ATP synthetase, subunit 8, and subsequences, and combinations thereof.

The phrase “nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Two nucleic acid sequences or polypeptides are said to be “identical” if the sequence of nucleotides or amino acid residues, respectively, in the two sequences is the same when aligned for maximum correspondence as described below. The term “complementary to” is used herein to mean all of a first sequence is complementary to at least a portion of a reference polynucleotide sequence.

Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman PNAS USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The percent identity between two sequences can be represented by any integer from 25% to 100%. More preferred embodiments include at least: 25%, 30%, 35%, 40%, 45%, 50%, 55%, 60%, 65%, 70%, 75%, 80%, 85%, 90%, 95%, or 99%.

One example of an algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, PNAS USA 89:10915 (1989)) alignments (B) of 50, expectation (E) of 10, M=5, N=−4, and a comparison of both strands.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Mixed nucleotides are designated as described in e.g. Eur. J. Biochem. (1985) 150:1.

### III. METHODS OF THE INVENTION

One embodiment of the present invention provides methods of amplifying, detecting, and/or measuring contaminating DNA (e.g., ruminant and non-ruminant) of less than about 100 base pairs (e.g., about 60 to about 100 base pairs, about 65 to about 95 base pairs, about 70 to about 90 base pairs, or about 75 to about 85 base pairs) in samples (e.g., ruminant feed, pet food, cosmetics, human food, and nutraceuticals). Target contaminating DNA (e.g., ruminant and non-ruminant) sequences of particular interest include mitochondrial DNA sequences and non-mitochondrial DNA sequences. Suitable mitochondrial DNA sequences include, for example, sequences encoding: cytochrome c, cytochrome b, 12S RNA, ATPase subunit 8, ATPase subunit 6, ATP synthetase, subunit 8, and subsequences and combinations thereof.

A. Nucleic Acid Extraction

Nucleic acids can be extracted from the sample using any method known in the art and/or commercially available kits. For example, the methods set forth in Sawyer et al., Foodborne Pathog Dis. 1(2):105-13 (2004) and Rensen et al., Foodborne Pathog Dis. 2005 Summer; 2(2):152-9 (2005) which employ Whatman FTA cards can be used to extract nucleic acids. Other suitable methods of extracting nucleic acids include guanidine isothiocyanate extraction as described in Tartaglia et al., J. Food Prot. 61(5):513-518 (1998); chelex extraction as described in Wang et al., Mol. Cell. Probes 14:1-5 (2000); extraction from Whatman paper as described in U.S. Pat. No. 5,496,562; extraction from cellulose based FTA filters as described in Orlandi and Lampe, J. Clin. Microbiology, 38(6): 2271-2277 (2000) and Burgoyne et al., 5th International Symposium on Human Identification, 1994 (Hoenecke et al., eds.) can be used to extract nucleic acids from the samples. In addition, the Neogen Kit (Neogen Catalog No. 8100), the Qiagen Stool Kit (Qiagen Catalog No. 51504), and the Qiagen Plant Kit (Qiagen Catalog No. 69181) can be used to extract nucleic acids from a sample.

In a preferred embodiment, cellulose based FTA cards (e.g., Whatman Catalog Nos. WB120055; WB120056; WB120205; WB120206; WB120208; WB120210) are used to extract nucleic acid. Samples diluted in an appropriate buffer are applied to the FTA card and allowed to dry. The FTA cards may comprise compounds that lyse cell membranes and denature proteins. DNA is captured within the matrix of the FTA cards and is stable at room temperature for up to 14 years. The FTA cards may comprise compounds that lyse cell membranes and denature proteins. For extraction of nucleic acids for PCR analysis of the sample (e.g., animal feed, human food, a vaccine, a cosmetic, or a nutraceutical), a punch (e.g., a 1-2 mm punch) is taken from the FTA card and the FTA card is washed according to manufacturer's instructions. The washed punch can then either be placed directly into a PCR reaction or the DNA can be eluted from the punch using any method known in the art. Liquid samples can be applied directly to the card without pre-processing. More complex samples (e.g., solid samples) may require processing prior to application to the FTA card. Typically, about 1 μl to about 1000 μl, more typically about 2.5 to about 500 μl, more typically about 5 μl to about 250 μl, more typically about 7.5 μl to about 100 μl, most typically about 10 μl to 65 μl sample can be placed on the FTA card.

Basic texts disclosing the general methods of use in this invention include MOLECULAR CLONING: A LABORATORY MANUAL (Sambrook et al. eds. 3d ed. 2001); PCR PROTOCOLS: A GUIDE TO METHODS AND Applications (Innis et al., eds, 1990); GENE TRANSFER AND EXPRESSION: A LABORATORY MANUAL (Kriegler, 1990); and CURRENT PROTOCOLS IN MOLECULAR BIOLOGY (Ausubel et al., eds., 1994)).

B. Amplification Reaction Components

1. Oligonucleotides

The oligonucleotides that are used in the present invention as well as oligonucleotides designed to detect amplification products can be chemically synthesized, using methods known in the art. These oligonucleotides can be labeled with radioisotopes, chemiluminescent moieties, or fluorescent moieties. Such labels are useful for the characterization and detection of amplification products using the methods and compositions of the present invention.

Typically, the target primers are present in the amplification reaction mixture at a concentration of about 0.1 μM to about 1.0 μM, more typically about 0.25 μM to about 0.9 μM, even more typically about 0.5 to about 0.75 μM, most typically about 0.6 μM. The primer length can be about 8 to about 100 nucleotides in length, more typically about 10 to about 75 nucleotides in length, more typically about 12 to about 50 nucleotides in length, more typically about 15 to about 30 nucleotides in length, most typically about 19 nucleotides in length. Preferably, the primers of the invention all have approximately the same melting temperature. Typically, the primers amplify a sequence of contaminating DNA (e.g., ruminant and non-ruminant) which exhibits high interspecies variation. Suitable target sequences include, for example, cytochrome B, cytochrome C, 12S RNA, ATPase subunit 8, ATPase subunit 6, ATP synthetase, subunit 8, and subsequences, and combinations thereof.

2. Buffer

Buffers that may be employed are borate, phosphate, carbonate, barbital, Tris, etc. based buffers. (See, U.S. Pat. No. 5,508,178). The pH of the reaction should be maintained in the range of about 4.5 to about 9.5. (See, U.S. Pat. No. 5,508,178. The standard buffer used in amplification reactions is a Tris based buffer between 10 and 50 mM with a pH of around 8.3 to 8.8. (See Innis et al., supra.).

One of skill in the art will recognize that buffer conditions should be designed to allow for the function of all reactions of interest. Thus, buffer conditions can be designed to support the amplification reaction as well as any subsequent restriction enzyme reactions. A particular reaction buffer can be tested for its ability to support various reactions by testing the reactions both individually and in combination.

3. Salt Concentration

The concentration of salt present in the reaction can affect the ability of primers to anneal to the target nucleic acid. (See, Innis et al.). Potassium chloride can added up to a concentration of about 50 mM to the reaction mixture to promote primer annealing. Sodium chloride can also be added to promote primer annealing. (See, Innis et al.).

4. Magnesium Ion Concentration

The concentration of magnesium ion in the reaction can affect amplification of the target sequence(s). (See, Innis et al.). Primer annealing, strand denaturation, amplification specificity, primer-dimer formation, and enzyme activity are all examples of parameters that are affected by magnesium concentration. (See, Innis et al.). Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.

5. Deoxynucleotide Triphosphate Concentration

Deoxynucleotide triphosphates (dNTPs) are added to the reaction to a final concentration of about 20 μM to about 300 μM. Typically, each of the four dNTPs (G, A, C, T) are present at equivalent concentrations. (See, Innis et al.).

6. Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 15 and 100 μl.

In some embodiments, a “hot start” polymerase can be used to prevent extension of mispriming events as the temperature of a reaction initially increases. Hot start polymerases can have, for example, heat labile adducts requiring a heat activation step (typically 95° C. for approximately 10-15 minutes) or can have an antibody associated with the polymerase to prevent activation.

7. Other Agents

Additional agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. (See, Innis et al. supra). Stabilizing agents such as gelatin, bovine serum albumin, and non-ionic detergents (e.g. Tween-20) are commonly added to amplification reactions. (See, Innis et al. supra).

C. Amplification

Amplification of an RNA or DNA template using reactions is well known (see, U.S. Pat. Nos. 4,683,195 and 4,683,202; PCR PROTOCOLS: A GUIDE TO METHODS AND APPLICATIONS (Innis et al., eds, 1990)). Methods such as polymerase chain reaction (PCR) and ligase chain reaction (LCR) can be used to amplify nucleic acid sequences of target DNA sequences directly from animal feed and animal feed components. The reaction is preferably carried out in a thermal cycler to facilitate incubation times at desired temperatures. Degenerate oligonucleotides can be designed to amplify target DNA sequence homologs using the known sequences that encode the target DNA sequence. Restriction endonuclease sites can be incorporated into the primers.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 15 seconds-2 minutes, an annealing phase lasting 10 seconds-2 minutes, and an extension phase of about 72° C. for 5 seconds-2 minutes.

In some embodiments, the amplification reaction is a nested PCR assay as described in, e.g., Aradaib et al., Vet. Sci. Animal Husbandry 37 (1-2): 13-23 (1998) and Aradaib et al., Vet. Sci. Animal Husbandry 37 (1-2): 144-150 (1998). Two amplification steps are carried out. The first amplification uses an “outer” pair of primers designed to amplify a highly conserved region of the target sequence. The second amplification uses an “inner” (i.e., “nested”) pair of primers designed to amplify a portion of the target sequence that is contained within the first amplification product.

Isothermic amplification reactions are also known and can be used according to the methods of the invention. Examples of isothermic amplification reactions include strand displacement amplification (SDA) (Walker, et al. Nucleic Acids Res. 20(7):1691 (1992); Walker PCR Methods Appl 3(l):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), and branched DNA signal amplification (bDNA) (see, e.g., Iqbal et al., Mol. Cell Probes 13(4):315 (1999)). In a preferred embodiment, rolling circle amplification (RCA) (Lisby, Mol. Biotechnol. 12(1):75 (1999)); Hatch et al., Genet. Anal. 15(2):35 (1999)) is used. Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS (hybrid capture system). Any amplification method known to those of skill in the art may be used with the methods of the present invention provided two primers are present at either end of the target sequence.

D. Detection of Amplified Products

Any method known in the art can be used to detect the amplified products, including, for example solid phase assays, anion exchange high-performance liquid chromatography, and fluorescence labeling of amplified nucleic acids (see MOLECULAR CLONING: A LABORATORY MANUAL (Sambrook et al. eds. 3d ed. 2001); Reischl and Kochanowski, Mol. Biotechnol. 3(1): 55-71 (1995)). Gel electrophoresis of the amplified product followed by standard analyses known in the art can also be used to detect and quantify the amplified product. Suitable gel electrophoresis-based techniques include, for example, gel electrophoresis followed by quantification of the amplified product on a fluorescent automated DNA sequencer (see, e.g., Porcher et al., Biotechniques 13(1): 106-14 (1992)); fluorometry (see, e.g., Innis et al., supra), computer analysis of images of gels stained in intercalating dyes (see, e.g., Schneeberger et al., PCR Methods Appl. 4(4): 234-8 (1995)); and measurement of radioactivity incorporated during amplification (see, e.g., Innis et al., supra).

In a preferred embodiment, dual labeled probes are used to detect the amplified DNA. Dual labeled probes include e.g., probes labeled with both a reporter and a quencher dye, which fluoresce only when bound to their target sequences and pairs of probes, each of which is labeled with a reporter and quencher day. In some embodiments, the dual labeled probes use fluorescence resonance energy transfer (FRET) technology in which probes labeled with either a donor or acceptor label bind within the amplified fragment adjacent to each other, fluorescing only when both probes are bound to their target sequences. Suitable reporters and quenchers include, for example, black hole quencher dyes (BHQ), TAMRA, FAM, CY3, CY5, Fluorescein, HEX, JOE, LightCycler Red, Oregon Green, Rhodamine, Rhodamine Green, Rhodamine Red, ROX, TAMRA, TET, Texas Red, and Molecular Beacons.

In another preferred embodiment, FRET probes and primers can be used to detect the contaminating DNA (e.g., ruminant and non-ruminant). One of skill in the art will appreciate that the primers and probes can conveniently be designed for use with the Lightcycler system (Roche Molecular Biochemicals). For example, a single set of primers (e.g., SEQ ID NOS: 1 and 2) and probes (SEQ ID NOS: 3 and 4) can conveniently be designed so that the DNA from multiple species of ruminants (e.g., cattle, goat, sheep, elk, deer, and the like) would amplify, and the probes would bind to all amplicons but with varying degrees of homology. The differences in homology result in distinct melting curve temperatures (Tm), each corresponding to an individual ruminant species.

In a preferred embodiment, RealTime PCR is used to detect target sequences. For example, in a preferred embodiment, Real-time PCR using SYBR® Green I can be used to amplify and detect the target nucleic acids (see, e.g., Ponchel et al., BMC Biotechnol. 3:18 (2003)). SYBR® Green I only fluoresces when bound to double-stranded DNA (dsDNA). Thus, the intensity of the fluorescence signal depends on the amount of dsDNA that is present in the amplified product. Specificity of the detection can conveniently be confirmed using melting curve analysis.

The amplification and detection steps can be carried out sequentially, or simultaneously.

### IV. KITS OF THE INVENTION

The present invention also provides kits for amplifying contaminating DNA (e.g., ruminant or non-ruminant). Such kits typically comprise two or more components necessary for amplifying contaminating DNA (e.g., ruminant and non-ruminant). Components may be compounds, reagents, containers and/or equipment. For example, one container within a kit may contain a first set of primers, e.g., SEQ ID NOS: 1 and 2; SEQ ID NOS: 5 and 6; SEQ ID NOS: 11 and 12; SEQ ID NOS: 15 and 16; or SEQ ID NOS: 19 and 20 and another container within a kit may contain a second set of primers, e.g., SEQ ID NOS: 1 and 2; SEQ ID NOS: 5 and 6; SEQ ID NOS: 11 and 12; SEQ ID NOS: 15 and 16; or SEQ ID NOS: 19 and 20 In addition, kits may contain a set of probes, e.g., SEQ ID NOS: 3 and 4; SEQ ID NOS: 7 and 8; SEQ ID NOS: 13 and 14; SEQ ID NOS: 17 and 18; or SEQ ID NOS: 21 and 22. In addition, the kits comprise instructions for use, i.e., instructions for using the primers in amplification and/or detection reactions as described herein.

### EXAMPLES

The embodiments of the present invention are further illustrated by the following examples. These examples are offered to illustrate, but not to limit the claimed invention.

### Example 1

### Materials and Methods

Preparation of cattle feed: Fifteen representative cattle feed samples were ground to a fine powder in a Wiley mill (Arthur H Thomas Co, Swedesboro, N.J., model 3375-E10) following official methods of analysis (see, e.g., JAOC, 16^{th} Edition published by AOAC, International Suite 400, 2200 Wilson Blvd., Arlington Va. 22201 1995, §§965.16 and 950.02). The feed samples were processed and placed on Whatman FTA® cards and stored at room temperature.

Feed Types:
1. Canola pellets
2. Cottonseed
3. Calf Maker
4. Calf Starter—Farmers Best
5. Dover dairy hominy with monesin
6. Dry citrus pulp
7. Grain pre-mix milk cows
8. High fat product (ADM)
9. Silage
10. Soy Bean meal
11. Soy plus heat treated soy bean meal
Mineral Mixes
1. CSP lactating cow mineral mix
2. Dover dairy milk cow mineral mix
3. lactating cow mineral mix
4. Vander kooi mineral mix

To confirm the absence of trace amounts of bovine products in the feeds, all feeds (unspiked and indicated as containing 0% bovine meat and bone marrow “BMBM”) were analyzed at the same time as the same feed spiked with rendered meat and bone meal. Rendered bovine meat and bone meal (BMBM) was mixed with the above seven feeds to produce feeds containing 2%, 1%, 0.5%, 0.2%, and 0.1% BMBM wt/wt. An unspiked sample of each feed (0% BMBM) was included as a negative control. One cattle feed (Feed 1) was selected to contain 0.05% and 0.01% BMBM and extracted only once.

European Rendered BMBM. A second sample set was also prepared in which thirty replicates of calf starter mix (Farmers Warehouse Co. Keys, Calif.) consisting of not less than 16% Crude Protein, not less than 2.5% crude fat, not more than 4.9% crude fiber, not more than 6.9% ash, not less than 0.65 calcium, not less than 0.4% phosphorous, not less than 0.6 ppm selenium, not less than 11000 IU/lb vitamin A, not more than 8% acid detergent fiber, was spiked with European rendered BMBM (Milan, Italy) at the 0.05-% w/w level. To obtain a concentration of 0.05-% w/w BMBM, 0.005 grams of European rendered BMBM was added to 10 g of each of the thirty replicates of the calf starter mix. The sample set also contained thirty replicates of a negative control in which no BMBM was added.

Processing offeed samples on Whatman FTA® cards. A Whatman punch was used to obtain a 2 mm disk containing the sample. The 2 mm disk was placed in a 1.5 mL sterile tube and 200 uL of Instagene (BioRad, Hercules, Calif., Cat#732-0630) was added. The samples were placed in a heating block at 56° C. for 30 min., then vortexed for 10 sec. The samples were then placed in a 100° C. heating block for 8 min., then vortexed for 10 sec and centrifuged at 12,000 rpm for 3 min. The supernate was removed and placed in a new sterile 1.5 mL tube for PCR analysis.

DNA extraction from calf starter mix spiked with European rendered BMBM. The 10 g feed samples were placed in a sterile 50 ml Falcon tube (Fisher Scientific, Pittsburgh, Pa.). A volume of 20 ml of cell lysis solution made up of 5M guanidinium isothiocyanate, 50 mM Tris-Cl, 25 mM EDTA, 0.5% Sarkosyl, 0.2M β-mercaptoethanol (Chakravorty and Tyagi, 2001) was added and the sample was vortexed. The sample was allowed to incubate at room temperature for 10 min. A volume of 65 μl of the cell lysis buffer was removed using a wide bore pipette tip and spotted onto a Whatman FTA® card (Whatman, Clifton, N.J., Cat #WB 12 0206) and allowed to dry at room temperature for 1 hour.

Bovine and ovine specific PCR primers and probes. The PCR primers and FRET probes were designed using the LightCycler Probe Design software version 2.0 (Roche Applied Science, Indianapolis, Ind.). The bovine primers and FRET probes were designed to target the bovine specific (mt-cyt-b) gene (NCBI Accession #D34635). The amplicon size is 97 bp. The forward primer sequence is GTGGACTATGGCAATTGCTA (SEQ ID NO: 1), the reverse primer sequence is CTGAGGCGGATTCTCAGTA (SEQ ID NO: 2), the fluorescein labeled probe sequence is ACCCGATTCTTCGCTTTCCATTT (SEQ ID NO: 3), and the LC-Red 640 labeled probe sequence is TCCTTCCATTTATC (SEQ ID NO: 4). The ovine primers and FRET probes were designed to target the ovine specific (mt-cyt-b) gene (NCBI Accession #AY858379). The amplicon size is 78 bp. The forward primer sequence is GCTACCCTCACCCGATT (SEQ ID NO: 5), the reverse primer sequence is GAGTAGGTGAACTATGGCGAG (SEQ ID NO: 6), the fluorescein labeled probe sequence is GCCTTTCACTTTAT (SEQ ID NO: 7), and the LC-Red 640 labeled probe sequence is TCCCATTCATCATCGC (SEQ ID NO: 8). All primers and FRET probes were then synthesized and labeled by Roche Applied Science.

Internal control PCR primers and probes: A previously described set of primers were used which target a region of the chloroplast RNA polymerase β-subunit (rpoβ) gene which is conserved amongst plants (Ozbek et al., FEMS Microbiology Letters 229:145-151 (2003)). The amplicon size is 192 bp. A set of FRET probes were then designed in order to target the 192 bp amplicon. The fluorescein labeled probe sequence is CCATAAGGGAATTTCTATATATGCCAGGACTATAA and the LC-Red 705 labeled probe sequence is TCAGATTGGGGGAGAAGGTCGGAATTAGC. All primers and FRET probes were then synthesized and labeled by Roche Applied Science.

Standard duplex FRET PCR protocol. After experimentally determining the optimum PCR conditions for the primers and FRET probes, the following standard LightCycler protocol was applied to all samples. All reactions were set up using the Roche LightCycler Multiplex DNA Master HybProbe kit (cat#04340019001). The PCR reaction was run at a final concentration of 0.3 uM ruminant forward primer, 0.7 uM ruminant reverse primer, 0.2 uM fluorescein labeled probe, 0.4 uM LC-Red 640 labeled probe, 0.25 uM rpoβ forward primer, 0.25 uM rpoβ reverse primer, 0.2 uM rpoβ fluorescein labeled probe, 0.2 uM rpoβ LC-Red 705 labeled probe and 1× LightCycler Multiplex DNA Master HybProbe mix. The DNA samples were added in 5 μl volumes to the reaction mixture for a total of 20 μl in each reaction. The conditions for cycling were 95° C. for 10 min. (denaturation and Taq. polymerase activation) followed by an amplification program of 50 cycles at 95° C. for 0 Sec., 42° C. for 12 sec., and 72° C. for 11 sec. LC-Red 640 was monitored in the F2 channel at the end of each 42° C. step and LC-Red 705 was monitored in the F3 channel. The amplification program was then followed with 1 melting cycle of 95° C. for 30 sec., 35° C. for 120 sec. and 80° C. for 0 sec with a transition rate of 0.1° C./sec. Continuous monitoring of fluorescent signal was carried out at the 80° C. step. The determination of a PCR positive result was made for bovine based on the presence of a melting curve in the F2 channel with a melting temperature (Tm) between 46° C. and 47° C. The determination of a PCR positive result was made for ovine based on the presence of a melting curve in the F2 channel with a melting temperature (Tm) between 48° C. and 49° C.

Evaluation of the ability to combine the bovine, ovine and internal control primer and probe sets to create a single PCR multiplex reaction. DNA from BMBM, LMBM and calf starter mix which was free of any MBM was mixed in solution. A PCR master mix containing primer and probe sets from bovine, ovine and rpoβ was prepared. PCR was performed on the DNA mixture samples. Electrophoresis was then performed on the PCR products. Each of the DNA bands (97 bp and 78 bp) were then excised using a sterile surgical scalpel. The Qiagen Qiaquick gel DNA kit (cat#28704) was then used in order to purify the PCR products from the agarose gel. The purified DNA samples were then sent to Davis Sequencing (Davis, Calif.) for direct sequencing. Direct sequencing was performed using the forward and reverse primer sets of bovine on the bovine amplicon (97 bp) and the forward and reverse primer sets of ovine on the ovine amplicon (78 bp). Sequence data from each of the PCR products were then analyzed, submitted in a blast search and compared against GenBank sequences of the mitochondrial cytochrome-b gene for bovine and ovine.

### Example 2

### Detection Of Ruminant DNA In Cattle Feed And Feed Ingredients Spiked With BMBM

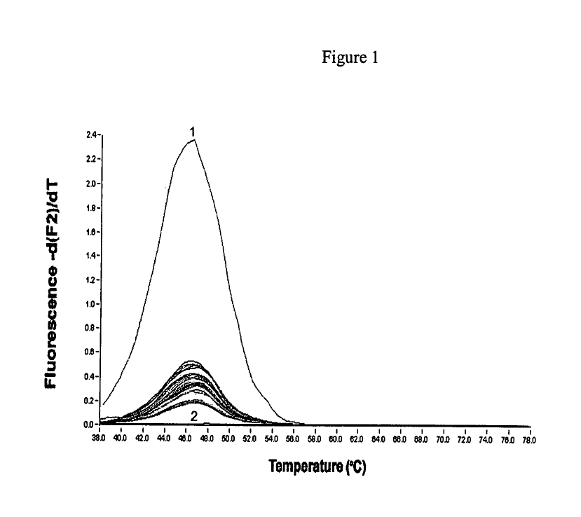

The bovine PCR assay with the internal control PCR reaction was able to detect BMBM at concentrations of 0.1-% w/w and 0.05-% on all archived feed samples tested over a period of six trials for each of the fifteen different types of cattle feed and feed ingredients. No false positive results occurred in any of the negative control samples. The internal control PCR reaction generated a positive PCR signal for all fifteen different types of cattle feed and feed ingredients. A representative melting curve analysis of cattle feed spiked at the 0.05-% level of BMBM is found in FIG. 1 where 1 denotes a PCR positive control—bovine DNA and 2 denotes a PCR negative control—5 ul of PCR grade water added to the 15 ul—reaction mixture. Thirty Tm melting curves representing PCR products from thirty DNA samples extracted from thirty samples of cattle feed and feed ingredients with BMBM at 0.05% wt/wt. The presence of a Tm melting curve represents a positive PCR result and positive detection of bovine contaminates in the cattle feed.

### Example 3

### Detection Of Ruminant DNA In Cattle Feed And Feed Ingredients Spiked With LMBM

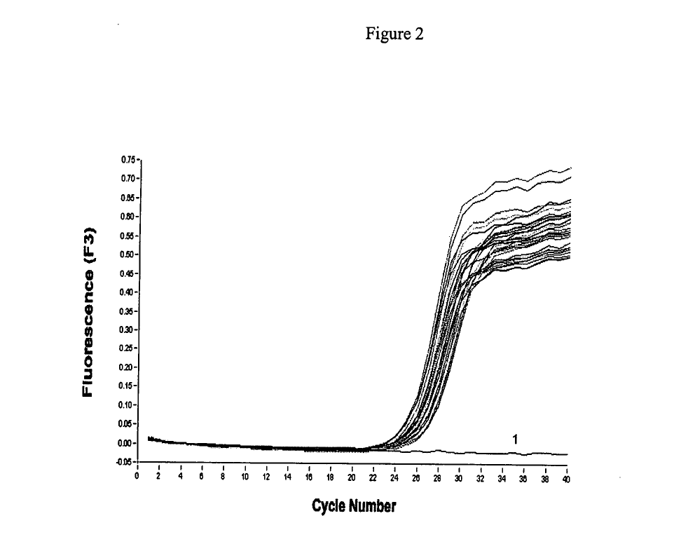

The ovine PCR assay with the internal control PCR reaction was able to detect LMBM at concentrations of 0.1% w/w and 0.05% on all archived feed samples tested over a period of six trials for each of the fifteen different types of cattle feed and feed ingredients. No false positive results occurred in any of the negative control samples. The internal control PCR reaction generated a positive PCR signal for all fifteen different types of cattle feed and feed ingredients. An example of the amplification curves generated by the internal control PCR reaction is shown in FIG. 2. PCR products from calf starter mix spiked at 0.1-% w/w BMBM, 0.1% w/w LMBM were visualized on a 4% agarose gel.

### Example 4

### Detection Of Ruminant DNA In Calf Starter Mix Spiked With European Rendered BMBM

The bovine PCR assay with the internal control PCR reaction was able to detect BMBM at the concentration of 0.05% w/w on all 30 samples of the calf starter mix. The mean average value in which the amplification signal crossed over the threshold limit was 29.2 cycles for 0.05% wt/wt BMBM in the calf starter mix.

### Example 5

### PCR Multiplex Reaction Targeting Bovine, Ovine and Internal Control

The PCR multiplex reaction amplified all three targets (bovine, ovine and rpoβ) in a single PCR reaction and generated three specific DNA bands of 192 bp (rpoβ), 97 bp (bovine) and 78 bp (ovine) that could be viewed in a 4-% agarose gel stained with ethidium bromide.

Direct sequencing of the PCR products generated by either the bovine or ovine primer sets was able to sequence 50% of the bovine amplicon and 50% of the ovine amplicon. Partial sequences were generated due to the fact that the described PCR primers for both bovine and ovine were used as the primers for direct sequencing of the amplicons. The partial sequences that were generated did corresponded to the correct mitochondrial cytochrome-b gene of either bovine (97 bp) or ovine (78 bp). Both BLAST searches and sequence alignment tools using gene bank resulted in a 100% match between the partial sequenced amplicons and regions of the bovine or ovine mitochondrial cytochrome b gene.

In this study we have designed and evaluated two species-specific PCR reactions with amplicons <100 bp in size, which are capable of detecting and identifying either bovine or ovine contaminates in cattle feed and feed ingredients including mineral mixes. Both species specific PCR assays are also duplex PCR reactions which incorporate a DNA extraction/PCR reaction internal control reaction to aid in the interpretation of PCR negative results. Both PCR assays use a previously described sample processing technique and DNA extraction method (Rensen et al, 2005) and were able to detect either BMBM or LMBM in cattle feed and feed ingredients. The bovine specific PCR assay can detect BMBM contaminates of 0.05-% w/w. The bovine specific PCR assay was also able to detect 0.05-% w/w of European rendered BMBM spiked into calf starter mix on all samples over a period of thirty trials. The ovine specific PCR assay can also detect LMBM contaminates of 0.05-% w/w. Both species specific PCR assays have the ability to detect MBM contaminates in either compound cattle feeds or mineral mixes without a decrease in sensitivity due to sample matrix inhibitory effects.

The study also incorporated samples that had been previously processed and archived on Whatman FTA® cards which allow for room temperature storage and archiving of samples for up to 14 years. All samples had been stored at room temperature for a minimum of one month and all samples did yield amplifiable DNA. The 2 mm punches were directly processed with Instagene (Chelex-100) which successfully removed PCR inhibitors and eluted the DNA from the 2 mm Whatman FTA® card punch into solution.

The PCR reactions were designed to be used with the older model of the LightCycler 1.5, which only allow for the use of two sets of FRET probes labeled with either LC-Red 640 or LC-Red 705. Due to the restrictions of the LightCycler 1.5, the detection limit had to be evaluated for bovine and ovine as two separate duplex PCR reactions. The results of combining the three PCR reactions targeting bovine, ovine and the internal control were shown to be effective, but could only be visualized on a 4-% agarose gel. Each of the FRET probe sets could also be labeled with different fluorescent molecules for simultaneous detection using an apparatus with six different fluorescent channels. The results would then be gathered in three different fluorescent channels, which would allow one to combine all primer and probe sets and target bovine, ovine and an internal control in a single PCR multiplex reaction.

Two real-time FRET probe based PCR assays to detect and discriminate between bovine and ovine contaminates in cattle feed utilizing sets of primers designed to target ruminant specific regions of the mitochondrial cytochrome b gene that are <100 bp in length have been developed and evaluated on fifteen different types of cattle feed and feed ingredients. Internal control PCR reaction targeting a region of the RNA polymerase η-subunit (rpoβ) gene which is conserved amongst plants has also been incorporated into both PCR reactions in order to simultaneously monitor the DNA extraction method and to test for the presence of PCR inhibitors. Both PCR assays were shown to work on previously processed feed samples that were archived on Whatman FTA® cards and stored at room temperature. The bovine specific PCR assay was also shown to detect both U.S. and European commercially rendered BMBM at the 0.05-% w/w contamination in compound feed. The newly developed PCR reactions targeting bovine and ovine DNA can be run as a single multiplex PCR reaction targeting bovine, ovine and rpoβ as an internal control, which could then be performed on the LightCycler 2.0.

### Example 6

### Goat-, Horse- and Pig-Specific Primers and Probes

Goat-, horse-, and pig-specific primers and probes have been designed that can be used to amplify and detect mitochondrial cytochrome-b gene sequences of about 100 bp or less. For example a goat-specific forward primer comprising the following sequence: ACCCGATTCTTCGCCTT (SEQ ID NO:11) and a goat-specific reverse primer comprising the following sequence: TGGGGTTGTTCGATCCTG (SEQ ID NO:12) can be used with the goat-specific primers comprising the following sequences: AGCCCTCGCCATAGTCCAC (SEQ ID NO: 13) or GCTCTTCCTCCAC (SEQ ID NO:14) can be used to amplify and detect an amplicon of 100 bp (from position 526-625 of the goat mitochondrial cytochrome b gene). A horse-specific forward primer comprising the following sequence: GCCACCCTTACCCGATT (SEQ ID NO: 15) and a horse-specific reverse primer comprising the following sequence: TAAATGTACGACTACCAGGGC (SEQ ID NO:16) can be used with horse-specific probes comprising the following sequences: GCTTTCCACTTCA (SEQ ID NO:17) or CTACCCTTCATCAT (SEQ ID NO:18) can be used to amplify and detect an amplicon of 75 bp (from position 517-591 of the equine mitochondrial cytochrome b gene). A pig-specific forward primer comprising the following sequence: TCACACGATTCTTCGCCTT (SEQ ID NO:19) and a pig-specific reverse primer comprising the following sequence: GTGCAGGAATAGGAGATGTACG (SEQ ID NO:20) can be used with pig-specific probes comprising the following sequences: TTTATCCTGCCATT (SEQ ID NO:21) or TCATTACCGCCCTCG (SEQ ID NO:22) to amplify and detect an amplicon of 80 bp (positions 524-603 of the pig mitochondrial cytochrome b sequence).

It is understood that the examples and embodiments described herein are for illustrative purposes only and that various modifications and changes in light thereof will be suggested to persons skilled in the art and are to be included within the purview of this application and are considered to be within the scope of the appended claims. All publications, patents, patent applications, and GenBank Accession Nos. cited herein are hereby incorporated by referenced in their entirety for all purposes.

What is claimed is:

1. A method of amplifying a contaminating DNA in a sample, said method comprising:
amplifying nucleic acid from the sample using a pair of primers designed to amplify a target DNA sequence of less than about 100 base pairs, thereby amplifying the contaminating DNA present in said sample.

2. The method of claim 1, wherein the target sequence is about 60 to about 100 base pairs.

3. The method of claim 1, wherein the target sequence is about 70 to about 90 base pairs.

4. The method of claim 1, wherein said contaminating DNA is a member selected from the group consisting of: cattle DNA, sheep DNA, goat DNA, equine DNA, swine DNA, and combinations thereof.

5. The method of claim 1, wherein said contaminating DNA comprises a mitochondrial DNA sequence.

6. The method of claim 5, wherein said mitochondrial DNA sequence encodes a member selected from the group consisting of: cytochrome c, cytochrome b, 12S RNA, ATPase subunit 8, ATPase subunit 6, ATP synthetase, subunit 8, and subsequences and combinations thereof.

7. The method of claim 5, wherein said mitochondrial DNA sequence encodes cytochrome b or a subsequence thereof.

8. The method of claim 1, wherein said pair of primers comprises sequences selected from the group consisting of: SEQ ID NOS:1 and 2; SEQ ID NOS: 5 and 6; SEQ ID NOS: 11 and 12; SEQ ID NOS: 15 and 16; and SEQ ID NOS: 19 and 20.

9. The method of claim 1, further comprising detecting said amplified contaminating DNA.

10. The method of claim 9, wherein detecting said amplified contaminating DNA comprises contacting said amplified contaminating DNA with an oligonucleotide probe.

11. The method of claim 10, wherein said oligonucleotide probe is labeled with a detectable label.

12. The method of claim 10, further comprising contacting said amplified contaminating DNA with a second oligonucleotide probe.

13. The method of claim 12, wherein detecting said amplified contaminating DNA comprises contacting said amplified contaminating DNA with a first oligonucleotide probe linked to a donor fluorophore and a second oligonucleotide probe linked to an acceptor fluorophore, wherein the donor fluorophore and the acceptor fluorophore interact to generate a detectable signal.

14. The method of claim 13, wherein said first oligonucleotide probe and said second oligonucleotide probe comprise the sequences set forth in SEQ ID NOS: 3 and 4; SEQ ID NOS: 7 and 8; SEQ ID NOS: 13 and 14, SEQ I) NOS: 17 and 18; or SEQ ID NOS: 21 and 22.

15. The method of claim 1, wherein said sample is a member selected from the group consisting of: an animal feed, an animal feed component, a cosmetic, a nutraceutical, a vaccine, a colloidal infusion fluid, or combinations thereof.

16. The method of claim 1, wherein said animal feed is cattle feed.

17. The method of claim 16, wherein said cattle feed comprises bovine tallow.

18. A kit for amplifying contaminating DNA, said kit comprising:
a pair of primers designed to amplify a target DNA sequence of less than about 100 base pairs, wherein the pair of primers is selected from the group consisting of SEQ ID NOS:1 and 2; SEQ ID NOS: 5 and 6; SEQ ID NOS: 11 and 12; SEQ ID NOS: 15 and 16; and SEQ ID NOS: 19 and 20; and
instructions for use.

19. The kit of claim 18, further comprising a first oligonucleotide probe linked to a donor fluorophore and a second oligonucleotide probe linked to an acceptor fluorophore, wherein the donor fluorophore and the acceptor fluorophore interact to generate a detectable signal.

20. The kit of claim 19, wherein said first oligonucleotide probe and said second oligonucleotide probe comprise: SEQ ID NOS: 3 and 4; SEQ ID NOS: 7 and 8; SEQ ID NOS: 13 and 14, SEQ ID NOS: 17 and 18; or SEQ ID NOS: 21 and 22.

21. An isolated nucleic acid comprising the nucleic acid sequence set forth in SEQ ID NOS:1, 2, 3, 4, 5, 6, 7, 8; 11, 12, 13, 14, 15, 16, 17, 18, 19, 20, 21, or 22.
